(19) United States
(12) Patent Application Publication
(10) Pub. No.: US 2025/0235174 A1
(43) Pub. Date: Jul. 24, 2025

(54) Securing a Dedicated Scan Operation

(71) Applicant: Siemens Healthineers AG, Forchheim (DE)

(72) Inventor: Stephan Biber, Erlangen (DE)

(73) Assignee: Siemens Healthineers AG, Forchheim (DE)

(21) Appl. No.: 19/033,783

(22) Filed: Jan. 22, 2025

(30) Foreign Application Priority Data
Jan. 22, 2024 (DE) 10 2024 200 547.9

(51) Int. Cl. A61B 6/00; G16H 30/40

(52) U.S. Cl. CPC A61B 6/5205; A61B 6/5229; G16H 30/40

(57) ABSTRACT

A method for dedicated medical imaging, including acquiring medical scan data from an investigation region of a patient, automatically carrying out an image recognition based on the acquired medical scan data, and automatically taking an enabling decision which relates to the medical image data generated based on the medical scan data, based on the image recognition.

### TECHNICAL FIELD

The disclosure relates to a method for dedicated medical imaging. The disclosure also relates to a monitoring facility. The disclosure further relates to a medical imaging system.

### BACKGROUND

Medical imaging systems, in particular, magnetic resonance imaging systems and computed tomography systems can be utilized both for whole-body imaging and also for the imaging of particular individual body regions or body parts.

A concept that includes the most efficient possible operation of medical imaging systems relates to the use of dedicated medical imaging systems, which have a limited area of use. Such a limited area of use is defined, for example, by the geometry and the dimensions of the interior of a medical imaging system. If a medical imaging system is to image only a particular group of body parts and never enable whole-body mapping, then this limitation can be achieved by way of the restriction of the dimensions of the patient opening of the medical imaging system. For example, an MR system (magnetic resonance imaging system) can be provided with a small bore diameter (i.e., a diameter of the patient opening) such that it does not permit the placement of the entire thorax or abdomen of a patient but only the placement of extremities, in particular, the knee, the foot, the wrist or the elbow and possibly also the head of a patient.

In particular, a magnetic resonance imaging system can also be configured such that the patient opening permits the placement of the entire patient, although it has only a small homogeneity volume of the applied B₀ field so that it is not possible to map the whole upper body or the entire head of the patient with sufficient quality, but only a part thereof and the magnetic resonance imaging system is thus also only suitable for the mapping of the extremities.

However, both limitations can be partially overcome. The limited dimensions of the patient opening can be overcome, for example, with particularly small or slim patients. A medical imaging system that is approved for imaging a very particular body part can easily be used for imaging another body part having similar dimensions.

For example, an MR system that should be used only for dental imaging can also be used for other body regions with particular limitations due to the specific protocols and contrasts specific thereto. It could be used, for example, for the mapping of extremities, even though the MR system is not configured for this use.

This incorrect use of the relevant medical imaging system leads to a reduced image quality and, thus, to a reduction in the quality of medical processes, in particular, the diagnosis and treatment of patients. A disadvantageous development of this type can, in an extreme case, be falsely attributed to the manufacturer, who has been unable to prevent such a “misuse.”

A restriction of a medical imaging system to dedicated applications can be achieved, for example, by way of the creation of an interface that limits the use of the medical imaging system to pre-determined applications. This limitation can be achieved in that the sequences and protocols provided for the imaging are not able to be freely parameterized and are also not freely selectable, but rather that only specific workflows and/or operational procedures are defined. In this way, a misuse of the medical imaging system can be impeded. For example, the workflows are optimized for dental imaging and can be used to only a limited extent for other body parts such as, for example, the elbow. However, users with minimal expectations can nevertheless not be prevented from using the medical imaging system for other body parts. For example, a scan unit that is intended for head imaging can also be “misused” for knee imaging.

The problem, therefore, exists in securing the utilization of dedicated medical imaging systems and preventing intentional or unintentional incorrect usage.

### SUMMARY

This object is achieved by way of a method for dedicated medical imaging, a monitoring facility, and a medical imaging system.

In the method according to the disclosure for dedicated medical imaging, medical scan data is acquired from an examination region of a patient. The medical scan data is received by a medical imaging system. The medical scan data can comprise raw data and/or image data. The medical scan data can comprise raw data and/or image data from the actual investigation but can also comprise so-called pre-scan data. The medical imaging system is configured by way of hardware specification and/or software specification for one or more particular types of imaging or for the mapping of one or more particular body regions.

Subsequently, an automated execution of an image recognition on the basis of the acquired medical scan data takes place. The image recognition comprises an establishment of content information that relates to the scan data or image data generated therefrom. The image recognition permits image regions and/or image subregions to be identified. Preferably the entire investigation region is recognized as a region of a particular body part, for example, as the knee region or a subregion of the investigation region is segmented and identified, for example, with a particular body part or organ.

As described later in detail, this automated image recognition can take place by way of an automated comparison with reference data, although it can also take place by way of a model trained on the basis of training data or another type of automated pattern recognition.

Finally, on the basis of the image recognition, an enabling decision is automatically taken, which relates to medical image data generated on the basis of the medical scan data. If the result of the image recognition is positive, i.e., it is determined that the generated medical image data corresponds in its content to the usage specifications of the medical imaging system, then the generated medical image data is released and can subsequently be displayed to the user. If, however, during the image recognition it is determined that the generated medical image data does not belong in terms of its content in the enabled field of use, it is either not enabled at all and is therefore not displayed to the user, or it is displayed with limitations, i.e. preferably only a subregion of the investigation region is displayed which agrees in its content with an enabled usage of the medical imaging system or the image data is displayed with reduced image quality so that it is usable with limitations only, for example as a scout image, but not for an evaluation or diagnosis. Advantageously, a dedicated imaging is achieved, which is restricted, with respect to content, to a predetermined limited field of use. In particular, the medical imaging system cannot be misappropriated. For example, an imaging system for imaging the arms cannot be used for imaging the head or the teeth. By way of example, a dentist “borrowing” an imaging system from a neighboring orthopedics specialist and not purchasing or renting an imaging system himself is prevented. Misdiagnoses attributable to inappropriate imaging, which results in qualitatively inadequate images, are also prevented.

The monitoring facility, according to the disclosure, has an input interface for receiving medical scan data from an investigation region of a patient from a medical imaging system. Part of the monitoring facility, according to the disclosure, is an image recognition unit for automatically carrying out an image recognition on the basis of the received medical scan data. The monitoring facility, according to the disclosure, also comprises an enabling unit for automatically taking an enabling decision that relates to the medical image data generated based upon the medical scan data on the basis of a result of the image recognition. The monitoring facility, according to the disclosure, shares the advantages of the method according to the disclosure for dedicated medical imaging.

The medical imaging system, according to the disclosure, preferably a magnetic resonance imaging system or a computed tomography system, has a scanning unit for acquiring medical scan data from a patient and a monitoring facility according to the disclosure with which the medical scan data recorded by the scan unit is analyzed and medical image data possibly generated on the basis thereof is enabled. The medical imaging system, according to the disclosure, shares the advantages of the monitoring facility according to the disclosure.

The medical scan data can comprise or be based upon, for example, magnetic resonance signals. Dependent upon the medical imaging system used for acquiring the medical scan data, however, other medical scan data that is typical for the respective medical imaging system is also conceivable.

A majority of the aforementioned components of the monitoring facility, according to the disclosure, can be realized entirely or partially in the form of software modules in a processor of a corresponding computing system, for example, by a computing system in a medical imaging system. A realization largely through software has the advantage that conventionally used computing systems can also easily be upgraded with a software update in order to operate in the manner according to the disclosure. In this respect, the object is also achieved by way of a corresponding computer program product with a computer program that is loadable directly into one or more computing systems, having program portions in order to perform the steps of the method according to the disclosure for dedicated medical imaging, in particular, the steps for automatically carrying out an image recognition and for automatically taking an enabling decision when the program is executed in the computing system or systems. Such a computer program product can comprise, apart from the computer program, additional components, if relevant, such as for example, documentation and/or additional components, including hardware components, for example, hardware keys (dongles, etc.) in order to use the software.

For transport to the computing system and/or for storage on or in the computing system, a computer-readable medium, for example, a memory stick, a hard disk drive, or another transportable or permanently installed data carrier can be used on which the program portions of the computer program which can be read in and executed by a computing system are stored. For this purpose, the computing system can have, for example, one or more cooperating microprocessors or such.

The dependent claims and the description below each contain particularly advantageous aspects and developments of the disclosure. In particular, the claims of one claim category can also be further developed similarly to the dependent claims of another claim category. In addition, in the context of the disclosure, the different features of different exemplary aspects and claims can also be combined to form new exemplary aspects.

As briefly described above, the medical scan data preferably comprises raw data and/or image data and/or pre-scan data. Pre-scan data can be generated with a lower resolution than the image data of the main recording. Such pre-scan data is preferably obtained by way of a navigator scan or during the recording of projection image data. Raw data comprises unprocessed scan data which is acquired from an investigation region during a medical imaging procedure. In the event that a computed tomography system is used as the imaging modality, raw data comprises projection data that is converted, by way of a filtered back projection, into image data. As previously mentioned, in the event that a magnetic resonance imaging system is used as the imaging modality, the unprocessed scan data included by the raw data can also comprise magnetic resonance signals and/or k-space data. Advantageously, all the data from the investigation region of a patient that is available for image recognition can be utilized so that a broad data basis is provided for image recognition. If the raw data is used for the image recognition, then an image reconstruction can possibly be dispensed with if it is already clear on the basis of the analysis of the raw data that the image recording is inappropriate and should not be displayed.

As has also been briefly indicated, the image recognition can take place on the basis of the following image recognition methods:
a comparison of the medical scan data with known reference scan data,
an automated pattern recognition on the basis of the acquired medical scan data, an image data recognition based upon an AI-based model (AI stands for artificial intelligence), which is applied to the medical scan data.

During a comparison, the medical imaging system can comprise a reference database that stores reference image data or reference scan data associated with specific body regions and/or organs that are to be identified in the context of the image recognition.

Automatic pattern recognition can be based on syntactic and/or statistical and/or structural pattern recognition.

Image data recognition on the basis of an AI-based model can be trained by way of training data to recognize image information on the basis of the scan data. Once the model has been trained, then for the image recognition itself, there is no longer a need to draw upon reference data.

Preferably, in the step of image recognition in the method, according to the disclosure, an organ situated in the investigation region or a body region comprising the investigation region is recognized. The recognized body region or the recognized organ can now advantageously be compared with data from a database that stores and specifies organs or body regions approved for imaging. The data in this database can be adapted to its specific application field before the handing-over of a medical imaging system to the customer. If the customer later acquires licenses because he wishes to extend his application field, further organs or body parts can then be stored in the database and thus licensed. In conjunction with the extension of the application field of the medical imaging system, specific software packages, which preferably comprise specific workflows, specific sequences, and specific contrast levels, can also be activated. A specific image filter can also be adapted. Advantageously, additional areas of use can be enabled for the customer for his medical imaging system as needed.

The enabling decision is thus preferably taken on the basis of specification data from a database that stores specification data for the enabling of approved investigation regions. Such specification data comprises information regarding specific subregions of the human body enabled for imaging, in particular, the head region, the trunk region, or the arms or legs. In detail, the specification data can also enable individual functional units or organs of the body and/or the named subregions, such as, for example, the jaw in the head region, the knee in the leg region, the liver, the heart, etc. as specific organs to be mapped.

In an aspect of the method according to the disclosure for dedicated medical imaging, on the basis of the image recognition, a classification of the acquired medical scan data and/or the generated medical image data takes place, and the enabling decision is taken dependent upon the classification of the acquired medical scan data and/or the generated medical image data. Thus, in particular, only generated medical image data with a predetermined classification is enabled. Advantageously, an allocation of the recorded medical image data to a known type of image data can take place via the classification, and the subsequent comparison of this type with approved types of image data can take place in order to find out whether the acquired image data is to be enabled or not.

Preferably, the classification therein comprises an allocation of the generated medical image data to a particular type of investigation region, in particular, to a particular body region, and the enabling decision is taken dependent upon the allocated type of the investigation region. Thus, in particular, only medical image data from one or more pre-determined body regions are enabled. Advantageously, only the position of the image recording in the body must be established as the basis for an enabling decision. The position can then be established, for example, by way of a comparison with image data, the position of which in the body is known.

Preferably, the enabling decision, also comprises an enabling of the medical image data with limited image quality dependent upon a result of the image recognition carried out. Rather than a binary decision, which comprises only a complete suppression of the enabling of image data or a complete enabling, apart from these two options, the enabling decision can also comprise the possibility of a decision that medical image data is enabled with only limited image quality. The enabling of medical image data with limited quality dependent upon a result of the image recognition carried out can, however, also be applied in the context of a binary decision scheme if, as possible options, only either a complete enabling of the image data or an enabling of medical image data with limited quality are available to choose from. Advantageously, on the basis of the medical image data with limited quality, an overview of a body region can be obtained, in particular, for the detection of a particular body structure, a particular organ or a particular functional unit. Advantageously, a user can obtain at least one scout image but without sufficient information to receive a reliable evaluation or diagnosis. Advantageously, the user is informed that the imaging has been carried out successfully without a “misuse” of the recorded image data taking place. For the reduction of the image quality, a downstream filter algorithm is preferably used with which the generated image data that have high quality are reduced in quality by way of a filter before they are output.

Preferably, the enabling with limited image quality comprises the enabling of the medical image data with reduced resolution and/or an enabling of the medical image data with a reduced matrix size. “Resolution” should be understood to mean the image sharpness and/or image size of a raster graphic. It is specified by the total count of image points or by the number of columns and rows in the raster graphic. The expression “matrix size” relates to the number of pixels or the number of columns and rows of an X-ray image or of a slice of a CT image or a magnetic resonance image. Advantageously, by way of the reduction of the image quality or the image information density, the usability of the image data is severely limited, and thus, a misuse of image data is prevented.

Preferably, a limited enabling takes place on the basis of the image recognition from only a pre-determined number of image recordings. For example, a classification of the generated medical image data takes place as image data from the extremities of a patient. If the user has obtained a license for only a restricted number of image recordings, for example, 100 image recordings of the extremities of a patient, then after the exhaustion of this limit, either no images at all or only images of reduced quality are output, and the user must first renew his license before he can continue to use his medical imaging system. By way of the identification of individual body regions or organs in the context of a so-called pay-per-use model, an individual use of the imaging system for a specific body region can also be specifically charged for.

Preferably, in the event that no enabling is granted, one of the following additional measures takes place:
there follows a termination of the imaging,
an error message is output,
the medical imaging system is deactivated for a pre-determined duration,
the patient is automatically removed from the medical imaging system.

Advantageously, it is unambiguously indicated to the user, either by communication or by an operational setting, that a misuse of the medical imaging system will not be accepted.

Preferably, the medical imaging system is limited by design measures to pre-determined body regions. Advantageously, a “misuse” of the medical imaging system can be at least impeded by way of its technical construction. For example, a dimensioning of the patient opening or a limitation of the dimensions of the region of the homogeneous B₀ magnetic field can restrict the area of use of the medical imaging system.

Therefore, the design measures preferably comprise one of the following:
a limitation of the dimensions,
a limitation of the homogeneous magnetic field region.

With the limitation of the dimensions, a “misuse” is hindered from the outset in that body parts for which the medical imaging system is not provided do not fit into the patient opening.

Through the limitation of the homogeneous magnetic field region, the investigation region is restricted so that extended body regions cannot be mapped completely with sufficient quality. These measures, which are also conventionally applied, can be combined with the method according to the disclosure in order to secure a dedicated use of a medical imaging system. In this way, “misuses” can be reduced from the outset so that, in the case of the limited dimensions of the patient opening, an unusable imaging is prevented in advance in many cases, so that an enhanced efficiency of the use of the medical imaging system is achieved. In the event of a limitation of a homogeneous magnetic field region, an inappropriate use can be notified on the basis of the analysis of the image data, but no image data with worsened quality is required to be specifically generated since the image data is already limited in its quality due to the inhomogeneity of the magnetic field so that a separate generation of image data of poorer quality can be dispensed with. The two measures create a certain synergy in this case. An additional filtration of the image data in order to make the image data unusable is no longer absolutely necessary.

### DESCRIPTION OF THE DRAWINGS

The disclosure will now be described again in greater detail using exemplary aspects, making reference to the accompanying drawings. In the drawings:

FIG. 1 shows a flow diagram that illustrates a method for dedicated medical imaging according to an exemplary aspect of the disclosure,

FIG. 2 shows a flow diagram that illustrates a method for dedicated medical imaging according to a second exemplary aspect of the disclosure,

FIG. 3 shows a flow diagram that illustrates a method for dedicated medical imaging according to a third exemplary aspect of the disclosure,

FIG. 4 shows a schematic representation of a monitoring facility according to an exemplary aspect of the disclosure, and

FIG. 5 shows a schematic representation of a magnetic resonance imaging system according to an exemplary aspect of the disclosure.

### DETAILED DESCRIPTION

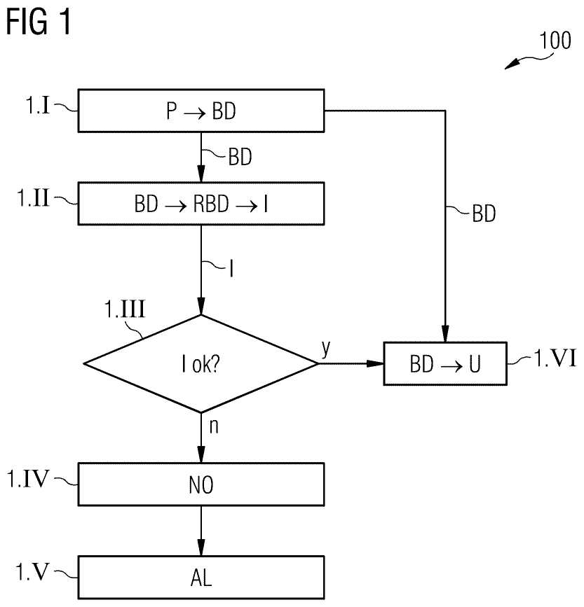

FIG. 1 shows a flow diagram 100 that illustrates a method for dedicated medical imaging according to an exemplary aspect of the disclosure.

In step 1.I, medical image data BD is acquired from a patient P with a medical imaging system, which is intended for a particular manner of imaging, for example, for dental imaging.

In step 1.II, a comparison of the acquired medical image data BD with previously known reference image data RBD is carried out automatically. In the exemplary aspect cited, the reference image data RBD comprises jaw mappings, which are automatically compared during the comparison with the currently acquired medical image data BD. During the comparison, the content I of the acquired medical image data BD.

In step 1.III, an automated enabling decision that concerns the medical image data BD is taken on the basis of a result of the comparison. That is, the newly acquired medical image data BD is only enabled if it has been identified as dental recordings. In the event that the content I of the medical image data BD is not included under the enabled use of the medical imaging system, this being indicated in FIG. 1 with “n”, control passes to step 1.IV, wherein on the basis of the comparison result, it is ascertained that no output NO of the medical image data BD takes place. In step 1.V, finally, a message AL is output that the imaging has taken place inappropriately and no medical image data BD is displayed to the user U.

In the event that the content I of the medical image data BD is included under the enabled use of the medical imaging system, this being indicated in FIG. 1 with “y”, control passes to step 1.VI, wherein the enabled medical image data BD is transferred to the user U.

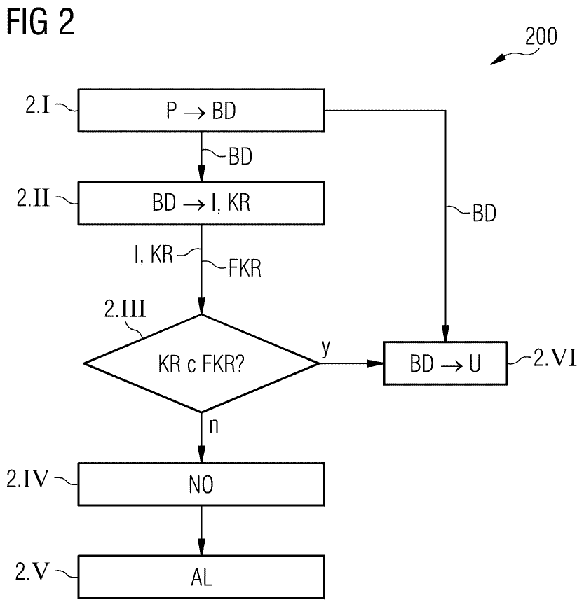

FIG. 2 shows a flow diagram 200 that illustrates a method for dedicated medical imaging according to a second exemplary aspect of the disclosure.

In step 2.I, medical image data BD is acquired from a patient P with a medical imaging system, which is intended for a particular manner of imaging, for example, for one or more enabled body regions FKR.

In step 2.II, the acquired medical image data BD is subjected to an automated pattern recognition, and image contents I of the image data BD are recognized. In particular, in step 2.II, it is established which body region KR or which organ is mapped by the medical image data BD.

In step 2.III, it is established whether the identified body region KR is included under a body region that is to be enabled FKR. In the event that the body region KR is not included under one of the body regions to be enabled FKR, this being indicated in FIG. 2 with “n”, a branch to step 2.IV takes place. In step 2.IV, it is established on the basis of the comparison result that no image data NO will be output. In step 2.V, a message AL is subsequently output that an inappropriate use of the magnetic resonance imaging system has taken place.

In the event that the body region KR is included under one of the body regions to be enabled FKR, this being indicated in FIG. 2 with “y”, a branch from step 2.III to step 2.VI takes place, wherein the acquired image data BD is output to the user U.

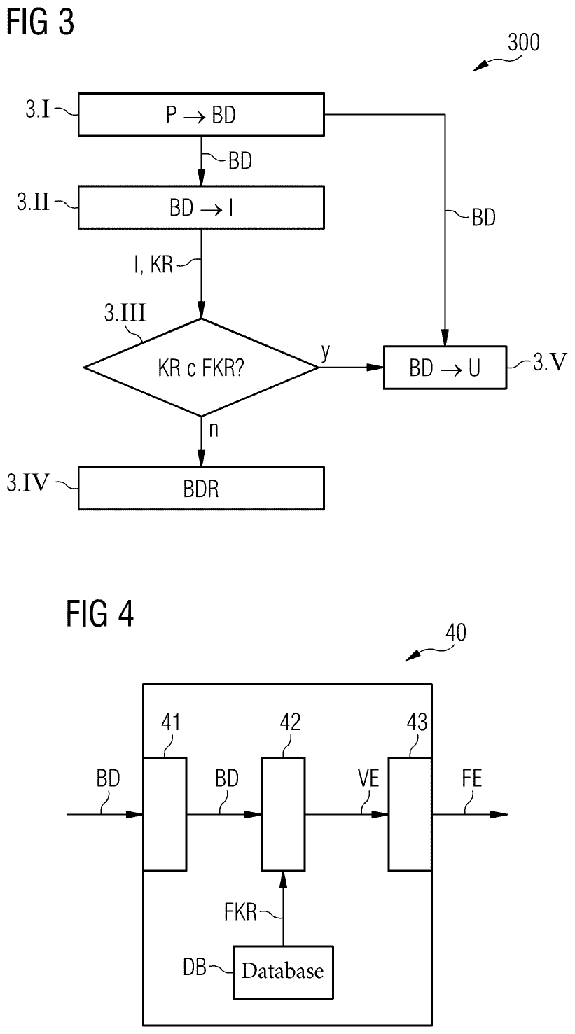

FIG. 3 shows a flow diagram 300 that illustrates a method for dedicated medical imaging according to a third exemplary aspect of the disclosure.

Similarly to the first and second exemplary aspects, in step 3.I, medical image data BD is acquired from a patient P.

In step 3.II, an AI-based image recognition is carried out on the basis of the acquired medical image data BD and contents I of the acquired medical image data BD, in particular, in the body regions KR contained in the medical image data BD, are established.

In step 3.III, it is established whether the body region KR is included under a body region that is to be enabled FKR. In the event that the body region KR is not included under one of the body regions to be enabled FKR, this being indicated in FIG. 3 with “n”, a branch from step 3.III to step 3.IV takes place. In step 3.IV, image data BDR is output with reduced resolution so that the user recognizes which body region KR has been acquired graphically, although the medical image data BD can only be further used to a limited extent.

In the event that the body region KR is included under one of the body regions to be enabled FKR, this being indicated in FIG. 3 with “y”, a branch from step 3.III to step 3.V takes place, wherein the acquired image data BD is output to the user U with full resolution.

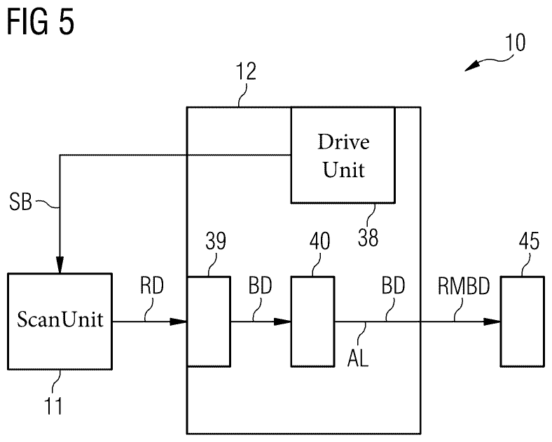

FIG. 4 shows a schematic representation of a monitoring facility 40 according to an exemplary aspect of the disclosure. The monitoring facility 40 has an input interface 41 for acquiring medical image data BD of a patient P from a magnetic resonance imaging system 10.

Also part of the monitoring facility 40, according to the disclosure, is an image recognition unit 42, which is configured to identify contents of the acquired medical image data BD and, in particular, to recognize and segment body regions KR. The image recognition unit 42 is also connected to a database DB, which stores enabled body regions FKR, information from which is compared with the recognized contents.

The monitoring facility 40, according to the disclosure, also has an enabling unit 43 for automatically taking an enabling decision FE, which relates to the medical image data BD on the basis of the comparison VE of the contents I recognized by the image recognition unit 42 with the data FKR from the database DB. The enabling decision FE is output by the enabling unit 43, for example, to a communication interface with a user, for example, a display unit.

FIG. 5 shows a schematic representation of a magnetic resonance imaging system 10 according to an exemplary aspect of the disclosure.

The magnetic resonance imaging system 10 has a scan unit 11 and a control facility 12. The control facility 12 comprises a drive unit 38, which is configured to control an imaging process of the scanning unit 11. The driving of the scan unit 11 takes place via control commands SB.

Also, part of the control facility 12 is reconstruction unit 39, which is configured on the basis of raw data RD, which the reconstruction unit 39 receives from the scan unit 11 to reconstruct image data BD.

The image data BD is transferred to a monitoring facility 40, which is also part of the control facility 12.If the image data BD is enabled by the monitoring facility 40, it is output via a display unit 45, in particular a screen, to the user. If the image data BD is not enabled, then alternatively, image data is output with a reduced resolution RMBD, and/or a message AL is communicated that the image data BD will not be output due to an inappropriate use.

Finally, it should again be noted that the methods and devices described above are merely preferred exemplary aspects of the disclosure and that the disclosure can be modified by a person skilled in the art without departing from the field of the disclosure to the extent that it is disclosed by the claims. For the sake of completeness, it should also be mentioned that the use of the indefinite article “a” or “an” does not preclude the relevant features from also being present plurally. Similarly, the expression “unit” does not preclude this consisting of a plurality of components that can possibly also be spatially distributed. Independent of the grammatical term usage, individuals with male, female, or other gender identities are included within the term.

1. A method for dedicated medical imaging, comprising:
acquiring medical scan data from an investigation region of a patient;
automatically performing an image recognition based on the acquired medical scan data; and
automatically taking an enabling decision which relates to medical image data generated based on the medical scan data, based on the image recognition.

2. The method as claimed in claim 1, wherein the medical scan data comprises data of a raw data type, an image data type, or a pre-scan data type.

3. The method as claimed in claim 1, wherein the image recognition comprises:
comparing the medical scan data with known reference scan data;
automatically performing pattern recognition based on the acquired medical scan data; or
performing image data recognition based on a model which is based upon artificial intelligence, which is applied to the medical scan data.

4. The method as claimed in claim 1, wherein in a context of the image recognition, an organ situated in the investigation region or a body region comprising the investigation region is recognized.

5. The method as claimed in claim 1, wherein the image recognition comprises classifying the acquired medical scan data and/or of the generated medical image data, and wherein the enabling decision is taken dependent upon the classification of the acquired medical scan data and/or the generated medical image data.

6. The method as claimed in claim 5, wherein the classifying comprises allocating the medical image data to a type of investigation region, and wherein the enabling decision is taken dependent upon the allocated type of the investigation region and only medical image data is enabled from one or more types of investigation regions.

7. The method as claimed in claim 1, wherein the enabling decision is taken based on specification data from a database which stores specification data for the enabling of approved investigation regions.

8. The method as claimed in claim 1, wherein the enabling decision comprises enabling the medical image data with limited image quality dependent upon a result of the image recognition carried out.

9. The method as claimed in claim 8, wherein the enabling with limited image quality comprises:
enabling the medical image data with a reduced resolution, and/or
enabling the medical image data with a reduced matrix size.

10. The method as claimed in claim 1, wherein a limited enabling of only a pre-determined number of image recordings takes place based on the image recognition.

11. The method as claimed in claim 1, wherein if no enabling is granted, one of the following additional measures takes place:
terminating of the medical imaging;
outputting an error message;
deactivating the medical imaging system used for the dedicated medical imaging for a pre-determined duration; or
automatically removing the patient from the medical imaging system.

12. A monitoring facility, comprising:
an input interface configured to receive medical scan data from an investigation region of a patient from a medical imaging system;
an image recognition unit configured to automatically perform an image recognition based on the received medical scan data; and
an enabling unit configured to automatically take an enabling decision which relates to medical image data generated based on the medical scan data, based on the image recognition.

13. A medical imaging system, comprising:
a scanning unit for generating medical image data from a patient; and
a monitoring facility as claimed in claim 12.

14. A non-transitory computer-readable medium comprising commands which, on execution by way of a computer cause the computer to carry out the steps of the method as claimed in claim 1.
